(19) United States
(12) Patent Application Publication
(10) Pub. No.: US 2026/0285016 A1
(43) Pub. Date: Sep. 24, 2026

(54) ENHANCED BIOPROCESSING FILM FOR SINGLE USE BIOCONTAINERS

(71) Applicant: Repligen Corporation, Waltham, MA (US)

(72) Inventors: John Saragosa, Melrose, MA (US); Niraj Chandarana, Franklin, MA (US)

(73) Assignee: Repligen Corporation, Waltham, MA (US)

(21) Appl. No.: 18/996,137

(22) PCT Filed: Aug. 1, 2023
(86) PCT No.: PCT/US2023/029164

§ 371 Date: Jan. 17, 2025

Related U.S. Application Data
(60) Provisional application No. 63/394,332, filed on Aug. 2, 2022.

(51) Int. Cl. B32B 27/08; A61J 1/10; A61J 1/14; B32B 25/08; B32B 27/30; B32B 27/32

(52) U.S. Cl. CPC B32B 27/08; A61J 1/10; A61J 1/1468; A61J 1/1475; B32B 25/08; B32B 27/304; B32B 27/306; B32B 27/32; B32B 2250/05; B32B 2250/24; B32B 2307/7244; B32B 2307/7376; B32B 2439/06

(57) ABSTRACT

A multilayer polymer film comprising an inner biomaterial contact layer and an outer layer, the outer layer comprising a polymer selected from a polyester elastomer copolymer, polyethylene terephthalate (PET), a nylon, polyethylene, polypropylene, a thermoplastic elastomer, and copolymers and mixtures thereof, wherein the melting temperature of the inner contact layer and the outer layer differ by 30-120° C.

### CROSS-REFERENCE TO RELATED APPLICATIONS

This application claims priority to U.S. Provisional application No. 63/394,332 filed Aug. 2, 2022, the contents of which are hereby incorporated in their entirety.

### FIELD OF THE INVENTION

The invention relates to disposable, single-use polymeric containers for containing a fluid and suitable for use in bioprocessing applications.

### BACKGROUND

Disposable, single-use containers for bioprocessing have been made from polymeric films converted into containers having a volume capacity ranging from 50 milliliters to as large as 5,000 liters. The typical conversion process from film into container utilizes various heat sealing or welding technologies to produce a sealed container. Such containers may find use in various aspects of bioprocessing, including as bioreactors, buffer and media storage containers, mixing containers, process sampling containers, and in final filling. Such containers have several advantages over traditional stainless steel or glass containers, disposable liners for such containers, and molded rigid or semi-rigid plastic containers for use in stainless steel bioreactors. For example, single-use polymeric containers can be made into various desired configurations, are generally less expensive than other options, and present a lower risk of cross-contamination and reduced need to maintain sterility due to their disposable nature. Various single-use containers have been described, for example in U.S. Pat. Nos. 6,391,404, 8,043,680, 8,556,111, 8,722,164, 10,899,114, and 11,110,684; and U.S. patents application Nos. 20150360450, 20170175066, 20210107261, and 20210347156. However, a lack of durability, including tendency to leak, remain obstacles impacting the use of such polymeric containers, especially for containers of larger sizes, and especially for three dimensional formed containers having a volume capacity greater than about 100 liters.

### BRIEF SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary

In aspects, the disclosure provides a multilayer polymer film comprising an inner biomaterial contact layer and an outer layer, the outer layer comprising a polymer selected from a polyester elastomer copolymer, polyethylene terephthalate (PET), a nylon, polyethylene, polypropylene, a thermoplastic elastomer, and copolymers and mixtures thereof, wherein the melting temperature of the inner contact layer and the outer layer differ by 30-120° C.

In aspects, the multilayer polymer film further comprises a gas barrier layer interposed between the inner biomaterial contact layer and the outer layer, a first tie layer interposed between the inner contact layer and the gas barrier layer, and a second tie layer interposed between the gas barrier layer and the outer layer. In aspects, the gas barrier layer comprises one or more materials selected from the group consisting of ethylene vinyl alcohol (EVOH), poly(vinylidene chloride) (PVDC), liquid crystal polymer (LCP), and nylon 6. In aspects, each tie layer comprises one or more materials selected from the group consisting of ethyl acrylate, ethyl acrylic acid, modified ethylene vinyl acetate, and polyethylene.

In aspects, the outer layer comprises a mixture of at least two polymers selected from a polyester elastomer copolymer, polyethylene terephthalate (PET), a nylon, polyethylene, polypropylene, a thermoplastic elastomer. In aspects, the outer layer comprises a mixture of at least two polyester elastomer copolymers.

In aspects, the inner biomaterial contact layer comprises a low density polyethylene. In aspects, the low density polyethylene is a linear low density polyethylene or an ultra low density polyethylene, or a mixture thereof. In aspects, the polyethylene is maleic anhydride grafted polyethylene.

In aspects, the inner biomaterial contact layer and the outer layer each have a different melt temperature.

In aspects, the film is an extruded polymer film. In aspects, at least 5 layers of the film are co-extruded to produce a single ply. In aspects, a single ply is from 0.20-0.50 millimeters thick.

In one aspect, provided is a multilayer polymer film including an inner biomaterial contact layer comprising about 70% weight percent of a low density polyethylene, an outer layer comprising about 5-10% weight percent of a polyester elastomer copolymer or a mixture of polyester elastomer copolymers having a melt temperature of from about 400-450° F., a gas barrier layer interposed between the inner biomaterial contact layer and the outer layer, a first tie layer interposed between the inner contact layer and the gas barrier layer, and a second tie layer interposed between the gas barrier layer and the outer layer, where the film has an oxygen transmission rate of less than about 0.5 cc/m2-day-atm at 70° F. under conditions in accordance with ASTM D-1307, where the film has a tear strength of at least about 8 LbF under conditions in accordance with ASTM D-1004, and where the melting temperature of the inner contact layer and the outer layer differ by 100-120° C.

The single use flexible film bag may also include end seals having a strength characterized by the ability to withstand at least about 2 pounds per square inch (psi) for at least about 12 months without failure.

In aspects, the disclosure also provides a single use flexible film bag comprising a body defining an interior space, the body comprising at least one polymeric sheet of the multilayer polymeric film described herein. In aspects, the body comprises at least 2-4 polymeric sheets.

In aspects, the bag further comprises at least one port assembly extending through an opening in the bag and communicating with the interior space of the body.

In aspects, the interior space of the body has a volume of from 50-5000 liters.

In aspects, the bag is sterile, optionally the bag is sterilized by gamma irradiation.

In aspects, the bag is suitable for containing a fluid. In aspects, the fluid comprises a biological material, optionally wherein the biological material is a cell or virus, and products thereof, including protein products and nucleic acid products.

Other technical features may be readily apparent to one skilled in the art from the following figures, descriptions, and claims.

### BRIEF DESCRIPTION OF THE DRAWINGS

Non-limiting aspects of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. In the figures, identical or nearly identical or equivalent elements are typically represented by the same reference characters, and similar elements are typically designated with similar reference numbers with redundant description omitted. For purposes of clarity and simplicity, not every element is labeled in every figure, nor is every element of each aspect shown where illustration is not necessary to allow those of ordinary skill in the art to understand the disclosure.

FIG. 1 is a schematic illustration of a multi-layer polymer film in accordance with an aspect of the present invention, showing the different layers.

FIG. 2 is a schematic illustration of a single use flexible container in accordance with an aspect of the present invention.

FIG. 3 a schematic illustration of a single use flexible container in accordance with an aspect of the present invention.

FIG. 4A is a photo of a representative flexible container in accordance with an aspect of the present invention which when inflated to 2 psi held its integrity for 17 months.

FIG. 4B is a photo of a representative flexible container in accordance with an aspect of the present invention which has been subjected to a burst test. The illustrated bag burst at 6 psi and showed failure away from end seals.

### DETAILED DESCRIPTION

The present invention provides multilayer polymer films for use in constructing single-use containers for bioprocessing, and related containers and methods. The polymer films described here have an enhanced film structure that provides an improved balance of strength, flexibility and robustness when converted into single-use containers. For example, single use flexible containers or bags manufactured from the polymer films described here demonstrate superior tensile yield, elongation, abrasion, tear, and outer layer durometer characteristics.

The multilayer polymer films described here comprise an inner biomaterial contact layer and an outer layer comprising a polymer selected from a polyester elastomer copolymer, polyethylene terephthalate (PET), a nylon, polyethylene, polypropylene, a thermoplastic elastomer, and copolymers and mixtures or blends of the foregoing. The terms “mixtures” and “blends” are used interchangeably herein. In aspects, the outer layer functions, in part, as a thermal barrier. In aspects, the outer layer comprises a polyester elastomer copolymer, which may also be referred to as a thermoplastic copolyester, or by the abbreviations TPC or TPE. In aspects, the outer layer comprises or consists essentially of a blend of two polyester elastomer copolymers. In aspects, the melting temperature of the outer layer is between 300-450 F.

In aspects, the multilayer polymer film further comprises a gas barrier layer interposed between the inner biomaterial contact layer and the outer layer, a first tie layer interposed between the inner contact layer and the gas barrier layer, and a second tie layer interposed between the gas barrier layer and the outer layer.

In aspects, the multilayer polymer film comprises from 5 to 11 layers. In aspects, at least 5 layers of the film are co-extruded to produce a single ply.

In aspects the multilayer polymer film is from 0.20-0.50 millimeters (mm) thick.

In aspects, the inner biomaterial contact layer and the outer layer each have a different melt temperature, wherein the difference in melt temperatures is not so great as to preclude coextrusion of the inner contact layer and the outer layer. In aspects, the difference in melt temperature between the inner biomaterial contact layer and the outer layer is between 30-120° C. In accordance with this aspect, the difference in melt temperature between the inner contact layer and the outer layer provides for faster heat bonding cycles and eliminates the possibility of the contact layer being heat sealed to the outer layer, thereby providing for an improved container manufacturing process.

The multilayer polymer films described here may be produced by a method such as cast film co-extrusion, blown film co-extrusion, or extrusion laminate/adhesive. In aspects, the multilayer polymer films described here may be produced by a method comprising a co-extruded cast film to produce a single-ply. In aspects, multiple plies of film may be utilized to construct a single-use container.

The multilayer polymer films described here are stable to gamma irradiation and are therefore sterilizable. In aspects, a container comprising a multilayer polymer film described here is sterilized.

In aspects, the inner biomaterial contact layer is characterized as comprising low levels of contaminating materials that may be extractable or leachable from the polymer surface. In this context, the term “extractable” refers to compounds that migrate from the inner contact layer into upon exposure to a solvent, generally under conditions of time and temperature that are outside the limits of standard bioprocessing operating procedures. In contrast, the term “leachable” refers to compounds that may migrate from the inner contact layer under standard operating conditions.

In aspects, the outer layer of the multilayer polymer films described has superior resistance to abrasion, puncturing, cracking and tearing as measured by industry standard tests.

In aspects, the multilayer polymer film comprises an inner biomaterial contact layer; an outer layer, the outer layer comprising a polymer selected from a polyester elastomer copolymer, polyethylene terephthalate (PET), a nylon, polyethylene, polypropylene, a thermoplastic elastomer, and copolymers and blends thereof; a gas barrier layer interposed between the inner biomaterial contact layer and the outer layer; a first tie layer interposed between the inner contact layer and the gas barrier layer; and a second tie layer interposed between the gas barrier layer and the outer layer. In aspects, the outer layer comprises a blend of at least two polymers selected from a polyester elastomer copolymer, polyethylene terephthalate (PET), a nylon, polyethylene, polypropylene, a thermoplastic elastomer and the inner biomaterial contact layer comprises a low density polyethylene.

In aspects, the containers constructed from the multilayer polymer films of the invention are in the form of a flexible film bag, including a disposable single use bag suitable for holding a fluid, including a fluid comprising one or more biological materials such as living cells. In aspects, the container takes the form of a flexible film bag suitable for storing and/or processing biological materials, including use as bioreactor for culturing living cells, which may be animal, insect, plant, or microbial cells, including for example yeast, bacteria, and viruses. In aspects, the film bag comprises a body, the body forming an interior space and comprising at least one polymeric sheet of the multilayer polymeric film described herein. In aspects, the body comprises at least two, three, or four polymeric sheets. In aspects, the bag further comprises at least one port assembly extending through an opening in the bag and communicating with the interior space of the body. In aspects, the interior space of the body has a volume of from 50-5000 liters or from 1000-5000 liters, or from 2000-5000 liters.

The flexible film bags constructed of the multilayer polymer films described here advantageously comprise end seals of high strength such that the ends seals can withstand at least about 2 pounds per square inch (psi) for a period of time of at least 12-16 months at room temperature and humidity without failure.

The polymer films described here may be converted into single-use containers suitable for containing a fluid, as well as allowing a fluid to be removed from and/or returned to the container. In aspects, the container may be a bioprocess containment vessel, a bioreactor, a retentate vessel, a mixer bag, or the like. The containers described here may be produced by a method comprising film converting, single-use container converting, assembly, packaging, and deployment into a final container. Standard heat sealing techniques can be used, for example, constant heat, thermal impulse, RF welding, and induction heating processes.

The fluid solution to be contained and/or processed within the vessel may be any of a variety of fluid solutions in the biological, pharmaceutical, biopharmaceutical, biotechnological, bioprocess.

It is to be understood that the present disclosure is not limited to the particular aspects described in the drawings, which depict illustrative aspects. All apparatuses and systems and methods discussed herein are examples of apparatuses and/or systems and/or methods implemented in accordance with one or more principles of this disclosure. Each example of an aspect is provided by way of explanation and is not the only way to implement these principles but are merely examples. Thus, references to elements or structures or features in the drawings must be appreciated as references to examples of aspects of the disclosure and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one aspect can be used with another aspect to yield a still further aspect. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular aspects only and is not intended to be limiting beyond the scope of the appended claims. Unless defined otherwise, technical terms used herein are to be understood as commonly understood by one of ordinary skill in the art to which the disclosure belongs.

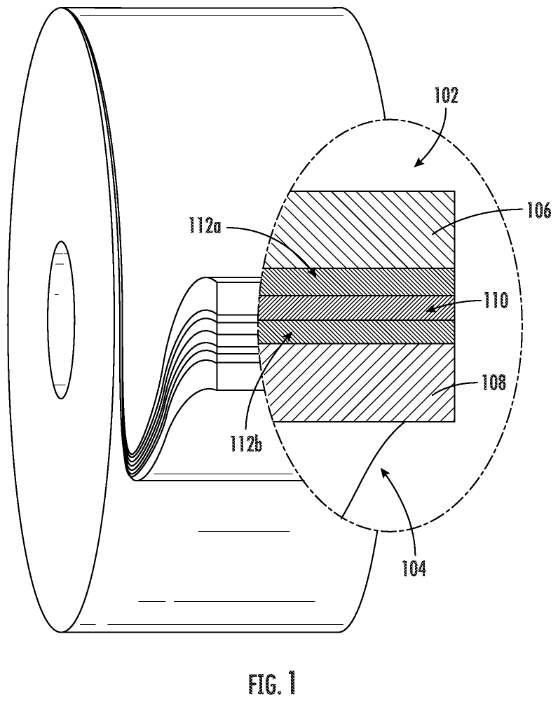

With reference to FIG. 1, shown is a schematic illustration of a polymeric film described herein with an exploded cutout view of the multiple layers and their arrangement with respect to the exterior side 102 and the fluid contact side 104 of the film. A gas barrier layer 110 is disposed between two tie layers, 112a and 112b; and each tie layer is positioned in contact with either the durable outer layer 106 which forms the exterior side of the film, or the fluid contact layer 108 which forms the interior of a flexible container manufactured with the polymeric film.

The outer layer 106 comprises a polymer selected from a polyester elastomer copolymer, polyethylene terephthalate (PET), a nylon, polyethylene, polypropylene, a thermoplastic elastomer, and copolymers and mixtures thereof. In some aspects, the melting temperature of the polymer or polymer mixture forming the outer layer 106 is from about 400-500° C. or from about 400-450° C. In some aspects, the outer layer 106 comprises a mixture of at least two polymers selected from a polyester elastomer copolymer, polyethylene terephthalate (PET), a nylon, polyethylene, polypropylene, a thermoplastic elastomer. In aspects, the outer layer comprises a polyester elastomer copolymer, or a mixture of at least two polyester elastomer copolymers. In aspects, the outer layer comprises a mixture of two polyester elastomer copolymers. The polymer mixtures may be, for example about a 50/50 wt % mixture, about a 60/40 wt % mixture, or about a 70/30 wt % mixture.

The gas barrier layer 110 comprises one or more materials selected from the group consisting of ethylene vinyl alcohol (EVOH), poly(vinylidene chloride) (PVDC), liquid crystal polymer (LCP), and nylon 6.

Each tie layer 112a, 112b comprises one or more materials selected from the group consisting of ethyl acrylate, ethyl acrylic acid, modified ethylene vinyl acetate, and polyethylene.

The inner layer 108, also referred to as the fluid contact layer or the biomaterial contact layer, comprises a low density polyethylene. In aspects, the low density polyethylene is a linear low density polyethylene or an ultra low density polyethylene, or a mixture thereof. In aspects, the polyethylene is maleic anhydride grafted polyethylene.

As discussed above, one feature of the multilayer polymer films described here is the differential melt temperatures between the inner layer 108 and the outer layer 106, which advantageously provides for faster heat bonding cycles during manufacture and also prevents the inner layer 108 from inadvertently being heat sealed to the outer layer 106. In some aspects, the melt temperatures of the inner and outer layers will differ by from about 30-120° C. or from about 50-120° C. or from about 100-120° C. In some aspects, the melt temperatures of the inner and outer layers will differ by at least 50° C. or at least 100° C.

In aspects, the film is an extruded polymer film comprising 5 layers as illustrated in FIG. 1, each layer being co-extruded to produce a single ply. In some aspects, a single ply is from 0.20-0.50 millimeters (mm) thick. In some aspects, a single 5-layer ply has a thickness of 0.20 mm, 0.25 mm, 0.35 mm, 0.45 mm, or 0.5 mm.

In some aspects, a multilayer polymer film in accordance with present disclosure is characterized by certain material properties including tear strength, oxygen transmission, abrasion resistance, puncture strength, and flex crack resistance, as determined in accordance with American Society for Testing and Materials (ASTM) standards. For example, in some aspects, the film is characterized by a tear strength of from about 7-9 pound-force (LbF) in accordance with ASTM D-1004 Standard Test Method for Tear Resistance (Braves Tear) of Plastic Film and Sheeting. In some aspects, the film is characterized by an oxygen transmission in the rate of 0.40-0.60 cubic centimeters per meter square (cc/m2) over 24 hours, or less than 1 cc/m2/day in accordance with ASTM D-1307. In some aspects, the film is characterized by an abrasion resistance of at least 500 or at least 600 cycles before failure in accordance with ASTM F-3300, a puncture strength of from about 12-16 LbF in accordance with ASTM F-1306, and/or a flex crack resistance over at least 800 or at least 900 cycles in accordance with ASTM F-392. Preferably, the film is characterized by a combination of the above material properties, for example a combination of each of the properties of tear strength, oxygen transmission, abrasion resistance, puncture strength, and flex crack resistance as described above.

In a specific example of a multilayer polymer film in accordance with present disclosure, the material properties were as shown in the following table.

TABLE 1
Material properties of a representative multilayer polymer film

| Test | Standard | Value | Unit |
|---|---|---|---|
| Tear strength | ASTM D-1004 | 8.5 | LbF |
| Oxygen transmission | ASTM D-1307 | 0.45 | cc/m2/24 hr |
| Abrasion resistance | ASTM F-3300 | 625 | cycles/failure |
| Puncture strength | ASTM F-1306 | 14.9 | LbF |
| Flex crack resistance | ASTM F-392 | 0 | pinholes/900 cycles |
| Coefficient of friction | ASTM D-1894 | 0.65 | (ratio) |

Also provided are single use flexible containers constructed from at least one polymeric sheet of a multilayer polymer film as described herein, which container may also be referred to as a “film bag”. The flexible containers described here comprise a body defining an interior space, the body comprising at least one polymeric sheet of the multilayer polymeric film described herein. In aspects, the body comprises at least 2-4 polymeric sheets. In some aspects, the body is formed from two or four polymeric sheets. In aspects, the bag further comprises at least one port extending through an opening in the bag and communicating with the interior space of the body. In aspects, the bag comprises from 2 to 4 ports. In aspects, the interior space of the body has a volume of from 50-5000 liters or from 100-5000 liters. In some aspects, the interior space of the body has a volume of about 100, about 250, about 500, about 750, or about 1000 liters. In some aspects, the interior space of the body has a volume of about 1,000 liters, about 2,000 liters, about 3,000 liters, about 4,000 liters, or about 5,000 liters.

In aspects, the bag is sterilized, for example by gamma irradiation.

In aspects, the bag is suitable for containing a fluid, such as fluid comprising a biological material, including for examples cells and/or cell products, including viral particles, and recombinant proteins or nucleic acids.

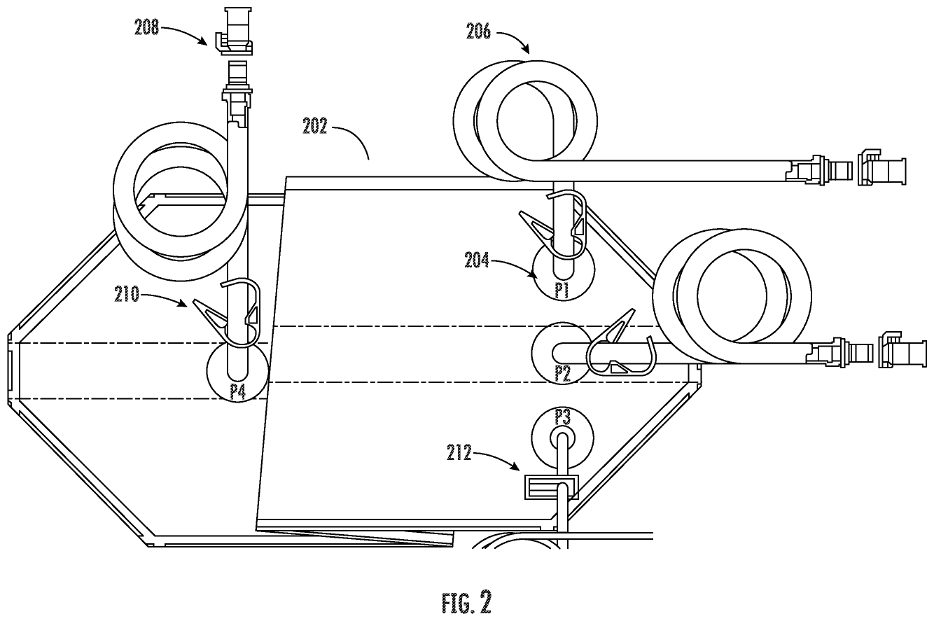

FIG. 2 is a schematic illustration of a flexible container in accordance with an aspect of the invention. The container is shown in a 2-dimensional view, which may also be considered a folded or collapsed view. The container is formed from four sheets of multilayer polymer film 202, which may be folded as shown for ease of packaging and assembly. Also shown are four ports 204 with associated tubing 206, clamps 210 and 212, which may be for example, pinch claims or slide clamps, and associated fittings 208.

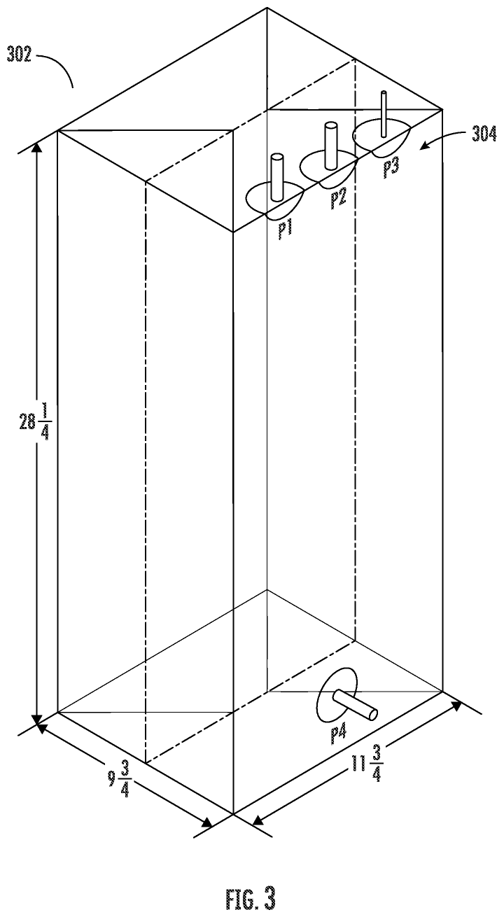

FIG. 3 is a schematic illustration of a flexible container in accordance with an aspect of the invention, shown in a 3-dimensional view with four ports 304. Dimensions are in inches. The illustrated container holds a volume of 50 liters but the basic design is the same for other volumes.

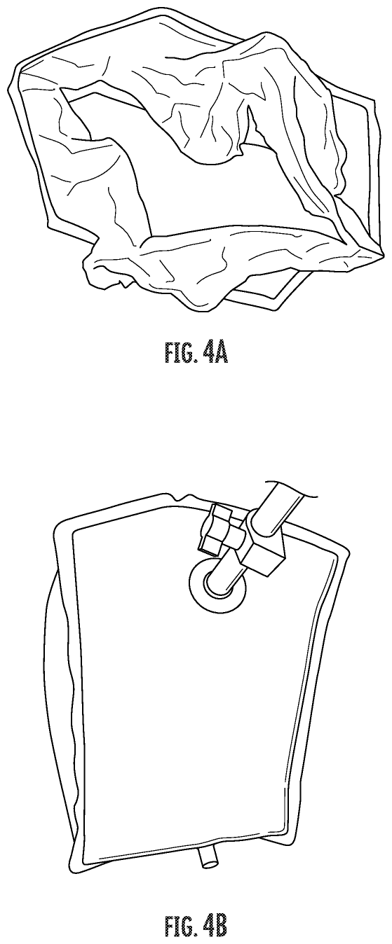

FIG. 4A is a photo of a representative flexible container made from a multi-layer polymer film as described herein. The container was inflated to 2 psi with air at room temperature (approximately 70° F.+/−10° F.) and monitored over a period of 17 months during which time no air was lost.

FIG. 4B is a photo of a flexible container similar to the container described in FIG. 4A which was subjected to a burst test. In this test, pressure is increased by 1 psi every 3 seconds. The bag illustrated here burst at 6 psi, which is well above the 2 psi that is a common hold pressure to ensure bag integrity. Importantly, this experiment showed that the failure points were at the end seals.

Reference in the present specification to “one aspect,” “an aspect,” “some aspects”, “other aspects”, etc. indicates that one or more particular features, structures, and/or characteristics in accordance with principles of the present disclosure may be included in connection with the aspect. However, such references do not necessarily mean that all aspects include the particular features, structures, and/or characteristics, or that an aspect includes all features, structures, and/or characteristics. Some aspects may include one or more such features, structures, and/or characteristics, in various combinations thereof. Moreover, references to “one aspect,” “an aspect,” “some aspects”, “other aspects”, etc. in various places in the specification are not necessarily all referring to the same aspect, nor are separate or alternative aspects necessarily mutually exclusive of other aspects. When particular features, structures, and/or characteristics are described in connection with one aspect, it should be understood that such features, structures, and/or characteristics may also be used in connection with other aspects whether or not explicitly described, unless clearly stated to the contrary. It should further be understood that such features, structures, and/or characteristics may be used or present singly or in various combinations with one another to create alternative aspects which are considered part of the present disclosure, as it would be too cumbersome to describe all of the numerous possible combinations and subcombinations of features, structures, and/or characteristics. Moreover, various features, structures, and/or characteristics are described which may be exhibited by some aspects and not by others. Similarly, various features, structures, and/or characteristics or requirements are described which may be features, structures, and/or characteristics or requirements for some aspects but may not be features, structures, and/or characteristics or requirements for other aspects. Therefore, the present disclosure is not limited to only the aspects specifically described herein, and the examples of aspects disclosed herein are not intended as limiting the broader aspects of the present disclosure.

It will be appreciated that common features in the drawings are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, certain features in one figure may be used across different figures and are not necessarily individually labeled when appearing in different figures.

Various features, aspects, or the like of a single use container or process described herein may be used independently of, or in combination, with each other. It will be appreciated that a container and/or system as disclosed herein may be embodied in many different forms and should not be construed as being limited to the illustrated aspects of the figures, such as described herein. Rather, these aspects are provided so that this disclosure will convey certain aspects of a container and/or process system formed in accordance with various principles of the present disclosure to those skilled in the art.

It should be understood that, as described herein, an “aspect” (such as illustrated in the accompanying Figures) may refer to an illustrative representation of an environment or article or component in which a disclosed concept or feature may be provided or embodied, or to the representation of a manner in which just the concept or feature may be provided or embodied. However such illustrated aspects are to be understood as examples (unless otherwise stated), and other manners of embodying the described concepts or features, such as may be understood by one of ordinary skill in the art upon learning the concepts or features from the present disclosure, are within the scope of the disclosure. In addition, it will be appreciated that while the Figures may show one or more aspects of concepts or features together in a single aspect of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one aspect can be used separately, or with one or more other features to yield a still further aspect. Thus, it is intended that the present subject matter covers such modifications and variations as come within the scope of the appended claims and their equivalents.

In view of the above, it should be understood that the various aspects illustrated in the figures have several separate and independent features, which each, at least alone, has unique benefits which are desirable for, yet not critical to, the presently disclosed vessel, system, and associated method. Therefore, the various separate features described herein need not all be present in order to achieve at least some of the desired characteristics and/or benefits described herein. Only one of the various features may be present in a vessel or system formed in accordance with various principles of the present disclosure. Alternatively, one or more of the features described with reference to one aspect can be combined with one or more of the features of any of the other aspects provided herein. That is, any of the features described herein can be mixed and matched to create hybrid designs, and such hybrid designs are within the scope of the present disclosure. Moreover, throughout the present disclosure, reference numbers are used to indicate a generic element or feature of the disclosed aspect. The same reference number may be used to indicate elements or features that are not identical in form, shape, structure, etc., yet which provide similar functions or benefits. Additional reference characters (such as letters, as opposed to numbers) may be used to differentiate similar elements or features from one another.

In the context of the present invention, the phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority but are used to distinguish one feature from another.

The term “about” when used in reference to a particular numerical value or a range of values encompasses values within plus or minus 10% of the value referred to, including the value itself.

Unless stated otherwise, all percentages, parts, ratios, etc., are by weight. Further, when an amount, concentration, or other value or parameter is given as either a range, preferred range or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range. When a component is indicated as present in a range starting from 0, such component is an optional component (i.e., it may or may not be present). When present an optional component may be at least 0.1 weight % of the composition or copolymer, unless specified at lower amounts

The terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus

The term “consisting essentially of” when used in reference to multilayer polymer film compositions described here indicates that substantially the only polymer(s) present in a composition are the polymer(s) recited. In this context, the term “substantially” means present in an amount greater than 95 weight % or greater than 99 weight %, based on total weight of the polymer composition. This term does not exclude the presence of conventional additives or polymeric carriers, provided that such polymeric carriers are present only in minor amounts of less than 5 weight % or less than 1 weight %. Any such minor amounts of additional polymeric materials do not change the basic and novel characteristics of the multilayer polymer film compositions described here.

As used herein, the term “copolymer” refers to polymers comprising copolymerized units resulting from copolymerization of two or more comonomers and may be described with reference to its constituent comonomers or to the amounts of its constituent comonomers such as, for example “a copolymer comprising ethylene and 15 weight % of methyl acrylate”. A description of a copolymer with reference to its constituent comonomers or to the amounts of its constituent comonomers means that the copolymer contains copolymerized units (in the specified amounts when specified) of the specified comonomers. Polymers having more than two types of monomers, such as terpolymers, are also included within the term “copolymer” as used herein. A dipolymer consists essentially of two copolymerized comonomers and a terpolymer consists essentially of three copolymerized comonomers. The term “consisting essentially of” in reference to copolymerized comonomers allows for the presence of minor amounts (i.e. no more than 0.5 weight %) of non-recited copolymerized units, for example arising from impurities present in the commoner feedstock or from decomposition of comonomers during polymerization.

Unless stated otherwise, all percentages, parts, ratios, etc., are by weight. Further, when an amount, concentration, or other value or parameter is given as either a range, preferred range or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range. When a component is indicated as present in a range starting from 0, such component is an optional component (i.e., it may or may not be present). When present an optional component may be at least 0.1 weight % of the composition or copolymer, unless specified at lower amounts.

The foregoing discussion has broad application and has been presented for purposes of illustration and description and is not intended to limit the disclosure to the form or forms disclosed herein. It will be understood that various additions, modifications, and substitutions may be made to aspects disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, aspects, or configurations for the purpose of streamlining the disclosure. However, it should be understood that various features of the certain aspects, aspects, or configurations of the disclosure may be combined in alternate aspects, aspects, or configurations. While the disclosure is presented in terms of aspects, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. The presently disclosed aspects are therefore to be considered in all respects as illustrative and not restrictive, the scope of the claimed subject matter being indicated by the appended claims, and not limited to the foregoing description or particular aspects or arrangements described or illustrated herein. In view of the foregoing, individual features of any aspect may be used and can be claimed separately or in combination with features of that aspect or any other aspect, the scope of the subject matter being indicated by the appended claims, and not limited to the foregoing description.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A multilayer polymer film comprising an inner biomaterial contact layer and an outer layer, the outer layer comprising a polymer selected from a polyester elastomer copolymer, polyethylene terephthalate (PET), a nylon, polyethylene, polypropylene, a thermoplastic elastomer, and copolymers and mixtures thereof, wherein the melting temperature of the inner contact layer and the outer layer differ by 30-120° C.

2. The multilayer polymer film of claim 1, further comprising a gas barrier layer interposed between the inner biomaterial contact layer and the outer layer, a first tie layer interposed between the inner contact layer and the gas barrier layer, and a second tie layer interposed between the gas barrier layer and the outer layer.

3. The multilayer polymer film of claim 1, wherein the outer layer comprises a mixture of at least two polymers selected from a polyester elastomer copolymer, polyethylene terephthalate (PET), a nylon, polyethylene, polypropylene, a thermoplastic elastomer.

4. The multilayer polymer film of claim 3, wherein the outer layer comprises a mixture of at least two polyester elastomer copolymers.

5. The multilayer polymer film of claim 1, wherein the inner biomaterial contact layer comprises a low density polyethylene.

6. The multilayer polymer film of claim 5, wherein the low density polyethylene is a linear low density polyethylene or an ultra low density polyethylene, or a mixture thereof.

7. The multilayer polymer film of claim 2 wherein the gas barrier layer comprises one or more materials selected from the group consisting of ethylene vinyl alcohol (EVOH), poly(vinylidene chloride) (PVDC), liquid crystal polymer (LCP), and nylon 6.

8. The multilayer polymer film of claim 2, wherein the tie layer comprises one or more materials selected from the group consisting of ethyl acrylate, ethyl acrylic acid, modified ethylene vinyl acetate, and polyethylene.

9. The multilayer polymer film of claim 8, wherein the polyethylene is maleic anhydride grafted polyethylene.

10. The multilayer polymer film of claim 1, wherein the film is an extruded polymer film.

11. The multilayer polymer film of claim 10, wherein at least 5 layers of the film are co-extruded to produce a single ply.

12. The multilayer polymer film of claim 11, wherein a single ply is from 0.20-0.50 millimeters thick.

13. The multilayer polymer film of claim 1, wherein the inner biomaterial contact layer and the outer layer each have a different melt temperature.

14. A multilayer polymer film comprising
an inner biomaterial contact layer comprising about 70% weight percent of a low density polyethylene,
an outer layer comprising about 5-10% weight percent of a polyester elastomer copolymer or a mixture of polyester elastomer copolymers having a melt temperature of from about 400-450° F.
a gas barrier layer interposed between the inner biomaterial contact layer and the outer layer,
a first tie layer interposed between the inner contact layer and the gas barrier layer, and
a second tie layer interposed between the gas barrier layer and the outer layer,
wherein the film has an oxygen transmission rate of less than about 0.5 cc/m2-day-atm at 70° F. under conditions in accordance with ASTM D-1307,
wherein the film has a tear strength of at least about 8 LbF under conditions in accordance with ASTM D-1004,
wherein the melting temperature of the inner contact layer and the outer layer differ by 100-120° C.

15. A single use flexible film bag comprising a body defining an interior space, the body comprising at least one polymeric sheet of the multilayer polymeric film of claim 1, wherein the bag is suitable for containing a fluid.

16. The single use flexible film bag of claim 15, wherein the body comprises at least 2-4 polymeric sheets of the multilayer polymeric film.

17. The single use flexible film bag of claim 15, comprising end seals having a strength characterized by the ability to withstand at least about 2 pounds per square inch (psi) for at least about 12 months without failure.

18. The single use flexible film bag of claim 15, wherein the bag further comprises at least one port assembly extending through an opening in the bag and communicating with the interior space of the body.

19. The single use flexible film bag of claim 15, wherein the interior space of the body has a volume of from 50-5000 liters.

20. The single use flexible film bag of claim 15, wherein the bag is sterile.
